(19) United States
(12) Patent Application Publication
(10) Pub. No.: US 2018/0271644 A1
(43) Pub. Date: Sep. 27, 2018

(54) CORNEA POSITIONING SYSTEM AND POSITIONING METHOD

(71) Applicant: SHENZHEN AINEAR CORNEA ENGINEERING CO., LTD., Shenzhen (CN)

(72) Inventors: Jinnan Zhang, Shenzhen (CN); Yongmei Yu, Shenzhen (CN)

(21) Appl. No.: 15/765,041

(22) PCT Filed: Sep. 30, 2016
(86) PCT No.: PCT/CN2016/101405

§ 371 Date: Mar. 30, 2018

(30) Foreign Application Priority Data
Sep. 30, 2015 (CN) 201510641337.5
Sep. 30, 2015 (CN) 201510646206.6

(51) Int. Cl. A61F 2/14; A61B 3/107; A61B 3/14; A61B 34/20; A61B 90/11; A61F 9/007; A61B 90/50; A61B 3/00; A61B 50/13

(52) U.S. Cl. CPC A61F 2/148; A61F 2/142; A61B 3/107; A61B 3/14; A61B 34/20; A61B 2017/00969; A61F 9/007; A61B 90/50; A61B 3/0008; A61B 50/13; A61B 2034/2057; A61B 90/11

(57) ABSTRACT

A cornea positioning system includes: a mechanical arm assembly, an image recognition system, a background assembly arranged opposite to the image recognition system, and a calculation positioning system connected to the image recognition system. The mechanical arm assembly is configured to move a corneal package body containing a cornea to-be-positioned to be between the image recognition system and the background assembly. The background assembly is configured to provide background light. The image recognition system is configured to obtain an image formed under action of the background light of cornea packaging body. The computing positioning system is configured to analyze and calculate the image obtained by the image recognition system acquire position information about the cornea.

### CROSS REFERENCE TO RELATED APPLICATIONS

This application is a national phase entry under 35 USC 371 of International Patent Application No. PCT/CN2016/101405 filed on 30 September 2016. which claims priority to Chinese patent Application No. 201510646206.6 filed with the Chinese Patent Office on Sep. 30, 2015, entitled “Cornea Positioning System and Cornea Positioning Method”, and Chinese patent Application No. 201510641337.5 filed with the Chinese Patent Office on Sep. 30, 2015, entitled “Cornea Automatically Positioning System and Method”, which are incorporated herein by reference in their entirety.

### TECHNICAL FIELD

The present disclosure relates to the field of medical instrument, and in particular to a cornea positioning system and a cornea positioning method.

### BACKGROUND

Corneal disease is the second blindness causing eye disease in the world and is increasing at a rate of 1.5-200 million cases per year. Corneal transplantation is currently the only effective method of treatment for corneal blindness, and corneal transplantation materials are mainly derived from corneal donation and corneal substitutes, such as acellular corneal stroma. Although the success rate of the corneal transplantation is quite high, and a rejection rate is very low, the recovery of eye vision after the corneal transplantation is very unsatisfactory. A main unsatisfactory manifestation is irregular astigmatism, as the corneal transplantation may not overcome the irregular astigmatism, and the current problem of the corneal transplantation is how to locate a cornea.

In the prior art, the positioning of the cornea is to check a corneal curvature and a corneal topography before a corneal transplantation operation to obtain position information of the cornea. As the positioning of the cornea in the prior art needs the placing of a corneal positioning ring to aid the positioning, this positioning procedure is cumbersome and a system error probability is high.

### SUMMARY

In one aspect, embodiments of the present disclosure provide a cornea positioning system, comprising: a mechanical arm assembly, an image recognition system, a background assembly disposed opposite to the image recognition system, and a computing positioning system connected to the image recognition system, wherein the mechanical arm assembly is configured to move a corneal package body containing a cornea to-be-positioned to be between the image recognition system and the background assembly;

The background assembly is configured to provide background light;

The image recognition system is configured to obtain an image, formed under action of the background light, of a corneal package body;

The computing positioning system is configured to analyze and calculate the image obtained by the image recognition system, to obtain position information of the cornea.

In a possible design, the system further comprises a main control system and a mechanical control system, wherein the mechanical control system is electrically connected with the mechanical arm assembly and configured to control the mechanical arm assembly to move the corneal package body containing the cornea to be between the image recognition system and the background assembly; the main control system is electrically connected with the image recognition system and the computing positioning system respectively, is configured to control the image recognition system to obtain the image, formed under the action of the background light, of the corneal package body when the mechanical arm assembly moves the corneal package body to be between the image recognition system and the background assembly, and is configured to control the computing positioning system to analyze and calculate the image obtained by the image recognition system to obtain the position information of the cornea.

In a possible design, the image recognition system comprises a camera, the background assembly comprises a positioning screen and a light source, and the light source is disposed behind the positioning screen relative to the image recognition system.

In a possible design, the center of the camera is disposed on a perpendicular center line of the positioning screen.

In a possible design, the mechanical arm assembly comprises a horizontally moving assembly, a vertically moving assembly and a clamping assembly, wherein the horizontally moving assembly is able to drive the vertically moving assembly and the clamping assembly to reciprocate in a horizontal direction parallel to a surface of the positioning screen, and the vertically moving assembly is able to drive the clamping assembly to reciprocate in a vertical direction parallel to the surface of the positioning screen.

In a possible design, the horizontally moving assembly comprises a first driving member, a horizontal guide rail, a horizontal lead screw disposed in the horizontal guide rail, and a first nut matching the horizontal lead screw, wherein the first nut is slidably connected with the horizontal guide rail, the first driving member is able to drive the horizontal lead screw to rotate, and the vertically moving assembly is fixedly connected with the first nut; and the vertically moving assembly comprises a second driving member, a vertical guide rail, a vertical lead screw disposed in the vertical guide rail, and a second nut matching the vertical lead screw, wherein the second nut is slidably connected with the vertical guide rail, and the second driving member is able to drive the vertical lead screw to rotate.

In a possible design, the clamping assembly comprises a support component, a first clamping component, a second clamping component, a resetting component, and a driving component, wherein a positioning window is disposed at each of central portions of the first clamping component and the second clamping component; the first clamping component is fixed on the support component, the first clamping component and the second clamping component are oppositely disposed, and the first clamping component and the second clamping component are connected via the resetting component therebetween; the driving component comprises a motor, an output shaft of the motor is connected with a swinging arm, one end of the swinging arm, away from the output shaft of the motor, abuts against the second clamping component, when the output shaft of the motor drives the swinging arm to swing toward the second clamping component, the swinging arm is able to push the second clamping component to rotate toward the first clamping component until the first clamping component touches the second clamping component, and when the output shaft of the motor stops driving the swinging arm to swing, the second clamping component rotate away from the first clamping component under action of the resetting component.

In a possible design, when the clamping assembly clamps the corneal package body to be between the image recognition system and the background assembly, a center position of the positioning window is positioned on the perpendicular center line of the positioning screen.

In a possible design, one end of the horizontal lead screw is provided with a rotary button, and when the rotary button is rotated, the horizontal lead screw is able to be driven to rotate, making the vertically moving assembly and the clamping assembly move in the horizontal direction.

In a possible design, the system also comprises a sample stage, wherein the sample stage is disposed below an initial position of the clamping assembly, a limiting groove is disposed on the sample stage, and the limiting groove is located directly under a gap between the first clamping component and the second clamping component when the first clamping component and the second clamping component are in a clamped state,

wherein the initial position is an initialization state of the clamping component preset on the mechanical arm assembly.

In a second aspect, embodiments of the present disclosure further provide a cornea positioning method, and the method comprises the following steps:

clamping a corneal package body containing a cornea to-be-positioned via a mechanical arm assembly in an initial position;

moving the corneal package body to be between an image recognition system and a background assembly via the mechanical arm assembly;

providing a background light via the background assembly, and obtaining an image, formed under action of the background light, of the corneal package body via the image recognition system; and

analyzing and calculating the image obtained by the image recognition system via a computing positioning system, to obtain position information of the cornea.

In a possible design, the mechanical arm assembly comprises a horizontally moving assembly, a vertically moving assembly and a clamping assembly, wherein the horizontally moving assembly is able to drive the vertically moving assembly and the clamping assembly to reciprocate in a horizontal direction parallel to a surface of a positioning screen; the vertically moving assembly is able to drive the clamping assembly to reciprocate in a vertical direction parallel to the surface of the positioning screen; the horizontally moving assembly comprises a first driving member, a horizontal guide rail, a horizontal lead screw disposed in the horizontal guide rail, and a first nut matching the horizontal lead screw, wherein the first nut is slidably connected with the horizontal guide rail, the first driving member is able to drive the horizontal lead screw to rotate, the vertically moving assembly is fixedly connected with the first nut; and the vertically moving assembly comprises a second driving member, a vertical guide rail, a vertical lead screw disposed in the vertical guide rail, and a second nut matching the vertical lead screw, wherein the second nut is slidably connected with the vertical guide rail, the second driving member is able to drive the vertical lead screw to rotate; and moving the corneal package body to be between the image recognition system and the background assembly via the mechanical arm assembly comprises:

switching on the second driving member, wherein the second driving member drives the vertical lead screw to rotate, to drive the clamping assembly to move vertically from the initial position in a direction parallel to the surface of the positioning screen, and switching off the second driving member when the corneal package body is at a same height as the image recognition system and the background assembly; and

switching on the first driving member, wherein the first driving member drives the horizontal lead screw to rotate, to drive the vertically moving assembly and the clamping assembly to move in the horizontal direction parallel to the surface of the positioning screen, and switching off the first driving member when the corneal package body is located between the image recognition system and the background assembly.

In a possible design, the clamping assembly comprises a support component, a first clamping component, a second clamping component, a resetting component, and a driving component,

wherein a positioning window is disposed at each of central portions of the first clamping component and the second clamping component, the first clamping component is fixed on the support component, the first clamping component and the second clamping component are oppositely disposed, the first clamping component and the second clamping component are connected via the resetting component therebetween, the driving component comprises a motor, an output shaft of the motor is connected with a swinging arm, and one end of the swinging arm, away from the output shaft of the motor, abuts against the second clamping component; and

clamping the corneal package body containing the cornea to-be-positioned via the mechanical arm assembly in the initial position, comprises:

placing and maintaining the corneal package body containing the cornea to-be-positioned between the first clamping component and the second clamping component when the motor is switched off and the second clamping component and the first clamping component are in an open state under action of the resetting component; and

switching on the motor, wherein the output shaft of the motor drives the swinging arm to swing toward the second clamping component, and the swinging arm pushes the second clamping component to rotate toward the first clamping component until the first clamping component touches the second clamping component.

In a possible design, the system further comprises a main control system and a mechanical control system,

wherein the mechanical control system is electrically connected with the mechanical arm assembly and configured to control the mechanical arm assembly to move the corneal package body containing the cornea to-be-positioned to be between the image recognition system and the background assembly;

the main control system is electrically connected with the image recognition system and the computing positioning system respectively, is configured to control the image recognition system to obtain an image, formed under the action of the background light, of the corneal package body when the mechanical arm assembly moves the corneal package body to be between the image recognition system and the background assembly, and is configured to control the computing positioning system to analyze and calculate the image obtained by the image recognition system to obtain position information of the cornea; and clamping the corneal package body containing the cornea to-be-positioned via the mechanical arm assembly in the initial position, comprises:

receiving, by the mechanical control system, a start instruction input by a user;

transmitting, by the mechanical control system, the start instruction input by the user to the main control system;

receiving, by the mechanical control system, a positioning instruction sent by the main control system according to the start instruction;

controlling, by the mechanical control system, the mechanical arm assembly to return to the initial position according to the positioning instruction;

sending, by the mechanical control system, a first signal to the main control system, wherein the first signal is configured to indicate that the mechanical arm assembly has returned to the initial position; and

receiving, by the mechanical control system, a first control signal sent by the main control system, and clamping, by the mechanical control system, the corneal package body containing the cornea in the initial position according to the first control signal.

In a possible design, the mechanical arm assembly comprises a horizontally moving assembly, a vertically moving assembly and a clamping assembly, wherein the horizontally moving assembly comprises a first driving member, a horizontal guide rail, a horizontal lead screw disposed in the horizontal guide rail, and a first nut matching the horizontal lead screw, wherein the first nut is slidably connected with the horizontal guide rail, the first driving member is able to drive the horizontal lead screw to rotate, and the vertically moving assembly is fixedly connected with the first nut; and the vertically moving assembly comprises a second driving member, a vertical guide rail, a vertical lead screw disposed in the vertical guide rail, and the second nut matching the vertical lead screw, the second nut is slidably connected with the vertical guide rail, and the second driving member is able to drive the vertical lead screw to rotate; and

controlling, by the mechanical control system, the mechanical arm assembly to return to the initial position according to the positioning instruction, comprises:

detecting, by the mechanical control system, a current value of the first nut on the horizontal guide rail and a current value of the second nut on the guide vertical rail;

controlling, by the mechanical control system, the first driving member to be switched on to drive the vertically moving assembly and the clamping assembly to move along the horizontal guide rail to be at a second initial value when the current value of the second nut on the vertical guide rail is a first initial value and the current value of the first nut on the horizontal guide rail is not the second initial value;

controlling, by the mechanical control system, the second nut to drive the clamping assembly to move along the vertical guide rail to be at the first initial value when the current value of the second nut on the vertical guide rail is not the first initial value and the current value of the first nut on the horizontal guide rail is the second initial value;

controlling, by the mechanical control system, the first nut to drive the clamping assembly to move along the horizontal guide rail to be at the second initial value, and controlling, by the mechanical control system, the first nut to drive the clamping assembly to move along the vertical guide rail to be at the first initial value when the current value of the second nut on the vertical guide rail is not the first initial value and the current value of the first nut on the horizontal guide rail is not the second initial value.

In a possible design, the clamping assembly comprises a support component, a first clamping component, a second clamping component, a resetting component, and a driving component, wherein a positioning window is disposed at each of central portions of the first clamping component and the second clamping component, the first clamping component is fixed on the support component, the first clamping component and the second clamping component are oppositely disposed, the first clamping component and the second clamping component are connected via the resetting component therebetween, the driving component comprises a motor, an output shaft of the motor is connected with a swinging arm, one end of the swinging arm, away from the output shaft of the motor, abuts against the second clamping component, when the output shaft of the motor drives the swinging arm to swing toward the second clamping component, the swinging arm is able to push the second clamping component to rotate toward the first clamping component until the first clamping component touches the second clamping component, and when the output shaft of the motor stops driving the swinging arm to swing, the second clamping component rotates away from the first clamping component under action of the resetting component; and

controlling, by the mechanical control system, the mechanical arm assembly to clamp the corneal package body containing the cornea in the initial position comprises: controlling, by the mechanical control system, the motor to be switched off, wherein the second clamping component and the first clamping component are in an open state under the action of the resetting component; placing and maintaining the corneal package body containing the cornea to be between the first clamping component and the second clamping component; and switching on, by the mechanical control system, the motor at a second time after a preset period, wherein the output shaft of the motor drives the swinging arm to swing toward the second clamping component, the swinging arm pushes the second clamping component to rotate toward the first clamping component until the first clamping component touches the second clamping component.

In a possible design, moving the corneal package body to be between the image recognition system and the background assembly via the mechanical arm assembly, comprises: controlling, by the mechanical control system, the second driving member to be switched on to drive the clamping assembly to slide downward a first distance along the vertical guide rail from the first initial value; controlling, by the mechanical control system, the second driving member to be switched off after the vertical guide rail slides downward the first distance making the corneal package body be at a same height as the image recognition system and the background assembly; controlling, by the mechanical control system, the first driving member to be switched on to drive the vertically moving assembly and the clamping assembly to move a second distance along the horizontal guide rail from the second initial position in a first direction, wherein the first direction is the direction of the horizontal guide rail toward a position between the image recognition system and the background assembly; and controlling, by the mechanical control system, the first driving member to be switched off after the first nut moves the second distance along the horizontal guide rail in the first direction making the corneal package body be located between the image recognition system and the background assembly.

In a possible design, providing the background light by the background assembly, and obtaining the image, formed under the action of the background light, of the corneal package body via the image recognition system, and analyzing and calculating the image obtained by the image recognition system via the computing positioning system, to obtain the position information of the cornea, comprises: controlling, by the main control system, the background assembly to provide the background light, and controlling, by the main control system, the image recognition system to obtain the image, formed under the action of the background light, of the corneal package body; and controlling, by the main control system, the computing positioning system to analyze and calculate the image obtained by the image recognition system to obtain the position information of the cornea.

In a possible design, after controlling, by the mechanical control system, the mechanical arm assembly to move the corneal package body to be between the image recognition system and the background assembly, before controlling, by the main control system, the computing positioning system to analyze and calculate the image obtained by the image recognition system to obtain the position information of the cornea, the method further comprises: controlling, by the main control system, the image recognition system and the mechanical control system making a center position of the positioning window of the clamping assembly be located on the perpendicular center line of the positioning screen.

In a possible design, controlling, by the main control system, the image recognition system and the mechanical control system to make the center position of the positioning window of the clamping assembly be located on the perpendicular center line of the positioning screen, comprises: sending, by the mechanical control system, a second signal to the main control system to indicate that the corneal package body is moved to be between the image recognition system and the background assembly after the mechanical control system controls the mechanical arm assembly to move the corneal package body to be between the image recognition system and the background assembly; sending, by the main control system, a second control signal to the image recognition system according to the second signal; detecting, by the image recognition system, a coordinate of the center position of the positioning window according to the second control signal; matching, by the image recognition system, the coordinate of the center position of the positioning window with a preset center coordinate; sending, by the image recognition system, deviation information between the coordinate of the center position of the positioning window and the preset center coordinate to the main control system when the coordinate of the center position of the positioning window does not match the preset center coordinate; sending, by the main control system, the deviation information to the mechanical control system; and adjusting, by the mechanical control system, the center position of the positioning window according to the deviation information.

In a possible design, the method further comprises: sending, by the computing positioning system, a third signal to the main control system when the computing positioning system analyzes and calculates the image obtained by the image recognition system and determines that there is no cornea related information in the image; sending, by the main control system, a third control signal to the mechanical control system according to the third signal; and controlling, by the mechanical control system, the mechanical arm assembly to return to the initial position according to the third control signal.

### BRIEF DESCRIPTION OF THE DRAWINGS

To describe the technical solution in embodiments of the present disclosure disclosure more clearly, the following briefly introduces the accompanying drawings required for describing the embodiments. Apparently, the accompanying drawings in the following description show merely some embodiments of the present disclosure, and those skilled in the art may still derive other drawings from these accompanying drawings without creative efforts.

FIG. 1 is a perspective view of a cornea positioning system provided by some embodiments of the present disclosure;

FIG. 2 is a top view of a cornea positioning system provided by some embodiments of the present disclosure;

FIG. 3 is a structural diagram of a background assembly provided by some embodiments of the present disclosure;

FIG. 4 is a structural diagram of a background assembly and an image recognition system being positioned provided by some embodiments of the present disclosure;

FIG. 5 is a structural diagram of a clamping assembly provided by some embodiments of the present disclosure;

FIG. 6 is a structural diagram of a sample stage provided by some embodiments of the present disclosure;

FIG. 7 is a structural diagram of a computing positioning system provided by some embodiments of the present disclosure;

FIG. 8 is a flowchart of a cornea positioning method provided by some other embodiments of the present disclosure;

FIG. 9 is a flowchart of another cornea positioning method provided by some other embodiments of the present disclosure;

FIG. 10 is a flowchart of another cornea positioning method provided by some other embodiments of the present disclosure;

FIG. 11 is a flowchart of a cornea positioning method provided by some further embodiments of the present disclosure;

FIG. 12 is a flowchart of another cornea positioning method provided by some further embodiments of the present disclosure;

FIG. 13 is a diagram of an operation interface of a mechanical control system according to some further embodiments of the present disclosure;

FIG. 14 is a diagram of another operation interface of a mechanical control system according to some further embodiments of the present disclosure;

FIG. 15 is a flowchart of another cornea positioning method provided by some further embodiments of the present disclosure; and

FIG. 16 is a flowchart of another cornea positioning method provided by some further embodiments of the present disclosure.

### DETAILED DESCRIPTION

The technical solutions in embodiments of the present disclosure will be clearly and completely described below with reference to the accompanying drawings in the embodiments of the present disclosure. Obviously, the described invcntion disclosure are only some of but not all of the embodiments of the present disclosure. All other embodiments obtained by those skilled in the art based on the embodiments of the present disclosure without creative efforts shall fall within the protection scope of the present invcntion disclosure.

In the description of the present disclosure, it is able to be understood that the orientation or positional relation indicated by the terms “center”, “upper”, “lower”, “back”, “left”, “right”, “vertical”, “horizontal”, “top”, “bottom”, “inner”, “outer” and the like are orientations or positional relations shown based on the accompanying drawings merely for convenience of description and simplifying description of the present disclosure, rather than indicating or implying that indicated devices or elements must have a particular orientation, be constructed and operated in a particular orientation, and therefore should not be construed as limiting the disclosure. Unless otherwise stated, in the description of the present disclosure, “a plurality of” means two or more. In the present disclosure, the X and Y axes are defined in two directions as: X—a horizontal abscissa axis; Y—a perpendicular vertical axis.

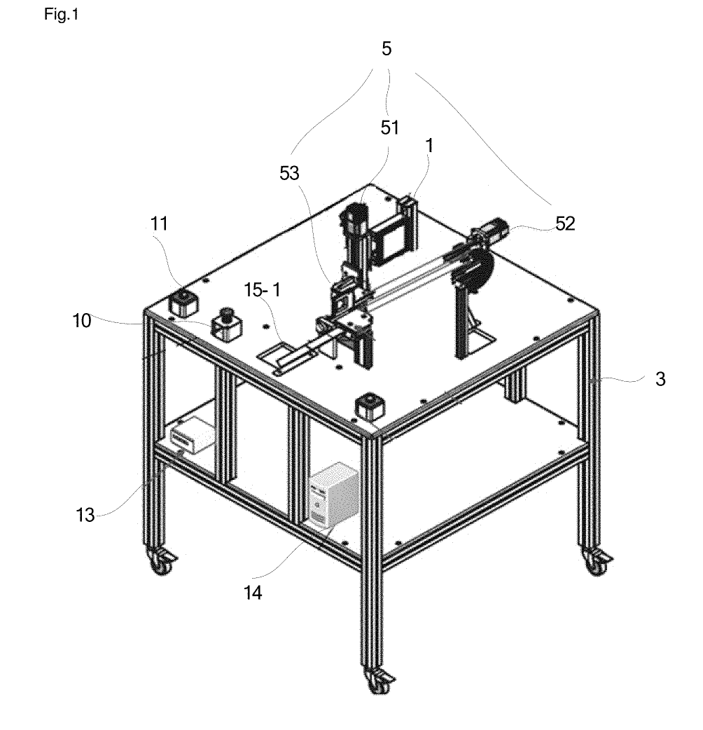

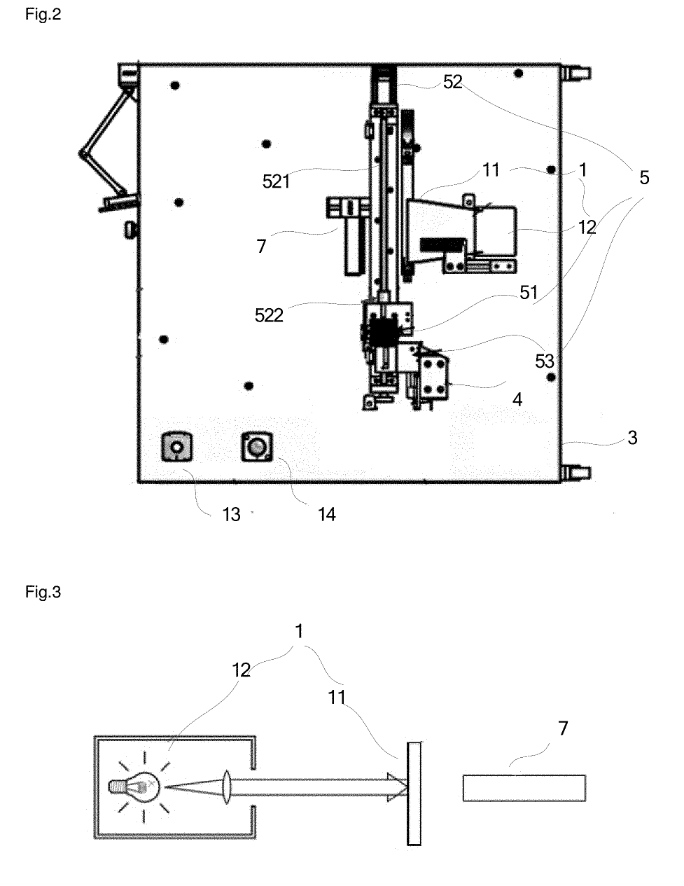

In one aspect, referring to FIG. 1 and FIG. 2, embodiments of the present disclosure provide a cornea positioning system including a mechanical arm assembly 5, an image recognition system 7, a background assembly 1 disposed opposite to the image recognition system, and a computing positioning system 8 connected to the image recognition system 7. The mechanical arm assembly 5 is configured to move a corneal package body (not shown in FIG. 1) containing a cornea to-be-positioned to be between the image recognition system 7 and the background assembly 1. The background assembly 1 is configured to provide background light. The image recognition system 7 is configured to obtain an image, formed under action of the background light, of the corneal package body. The computing positioning system 8 (not shown in the FIG. 1) is configured to analyze and calculate the image obtained by the image recognition system 7 to obtain position information of the cornea.

According to a cornea positioning system provided by embodiments of the present disclosure, when a cornea needs to be positioned, the cornea is able to be placed in a corneal package body, the corneal package body is moved to be between the image recognition system and the background assembly via the mechanical arm assembly, the background light is provided by the background assembly, the image, formed under the action of the background light, of the corneal package body is obtained via the image recognition system, and then, the computing positioning system is utilized to analyze and calculate the image obtained by the image recognition system to obtain the position information of the cornea. The cornea positioning system provided by embodiments of the present disclosure may not only improve the accuracy of cornea positioning but also reduce the labor of workers.

Embodiments of the present disclosure do not particularly limit the material, shape and size of the corneal package body. In some embodiments, in order to facilitate the background light provided by the background assembly passing through the corneal package body containing a cornea to-be-positioned to form an image, optionally, a transparent and homogeneous material with a smooth surface is adopted for the corneal package body in embodiments of the present disclosure, so that on the background light provided by the background assembly 1 is able to directly penetrate the cornea to-be-positioned in the corneal package body, to make an image of the cornea on the positioning screen 11.

Optionally, the cornea positioning system provided in embodiments of the present disclosure further comprises a main control system and a mechanical control system.

The mechanical control system is electrically connected with the mechanical arm assembly and configured to control the mechanical arm assembly to move the corneal package body containing the cornea to-be-positioned to be between the image recognition system and the background assembly.

The main control system is electrically connected with the image recognition system and the computing positioning system respectively. The main control system is configured to control the image recognition system to obtain the image, formed under the action of the background light, of the corneal package body when the mechanical arm assembly moves the corneal package body to be between the image recognition system and the background assembly. And the main control system is configured to control the computing positioning system to analyze and calculate the image obtained by the image recognition system to obtain the position information of the cornea. In this way, when an cornea needs to be positioned, the cornea to-be-positioned is able to be placed in a corneal package body, the mechanical arm assembly is controlled by the mechanical control system to move the corneal package body containing the cornea to be between the image recognition system and the background assembly, the background light is provided by the background assembly, an image, formed under the action of the background light, of the corneal package body is obtained via the image recognition system which is controlled by the main control system, and then the computing positioning system is controlled by the main control system to analyze and calculate the image obtained by the image recognition system to obtain the position information of the cornea. The cornea positioning system provided by embodiments of the present disclosure may not only improve the accuracy of the cornea positioning but also reduce the labor of workers.

To facilitate the mechanical arm assembly 5 being able to clamp tightly the corneal package body when in a clamped state, in some embodiments, a shape of the corneal package body is able to be designed according to a clamping shape of the clamping assembly 53 of the mechanical arm assembly 5. For example, the corneal package body is able to be designed into a shape which is clamped easily by the clamping assembly 53 of the mechanical arm assembly 5, which is not limited in embodiments of the present invcntion disclosure, as long as the mechanical arm assembly 5 is able to clamp tightly the corneal package body and drive it to move, when in the clamped state.

The thickness of the corneal package body shall be less than or equal to the maximum opening distance of the clamping assembly 53, in an open state, of the mechanical arm assembly 5, which is not limited in embodiments of the present disclosure.

The cornea in the corneal package body is not limited, and each cornea that needs to be positioned is able to be the cornea of the present disclosure.

The specific structure of the image recognition system 7 is not limited, as long as the image recognition system 7 is able to obtain the image formed by the background light of the background assembly passing through the corneal package body clamped by the mechanical arm assembly 5. In some embodiments, the image recognition system 7 in embodiments of the present disclosure is a camera. The camera is configured to scan the image formed by the background light of the background assembly passing through the corneal package body clamped by the mechanical arm assembly 5, to obtain the image, formed through the background light, of the cornea.

As shown in FIG. 3, further, the background assembly 1 comprises a positioning screen 11 and a light source 12. The light source 12 is disposed behind the positioning screen 11 relative to the image recognition system 7.

The material of the positioning screen 11 is not limited in embodiments of the present disclosure and is able to be selected according to a need. In order to accurately position the cornea in the corneal package body, exemplarily, in a practical use, a two-dimensional coordinate system is able to be disposed on the positioning screen 11. The X axis of the coordinate system is the lower frame edge of the positioning screen 11, and the Y axis thereof is the left frame edge, adjacent to the lower frame edge, of the positioning screen 11. Any position on the positioning screen 11 is able to be identified by a coordinate (x, y) in the coordinate system. For example, in a practical use, when the background light passes through the corneal package body clamped by the mechanical arm assembly 5, forming an image on the positioning screen 11, positions, A (x1, y1), B (x2, y2) . . . N (xn, yn) in the coordinate system, of all the pixel points in the image are recorded.

In embodiments of the present disclosure, the specific color of the light source 12 behind the positioning screen 11 is not limited, as long as the light source 12 is able to provide the background light making the image able to be formed on the positioning screen 11 when the background light passes through the corneal package body clamped by the mechanical arm assembly 5.

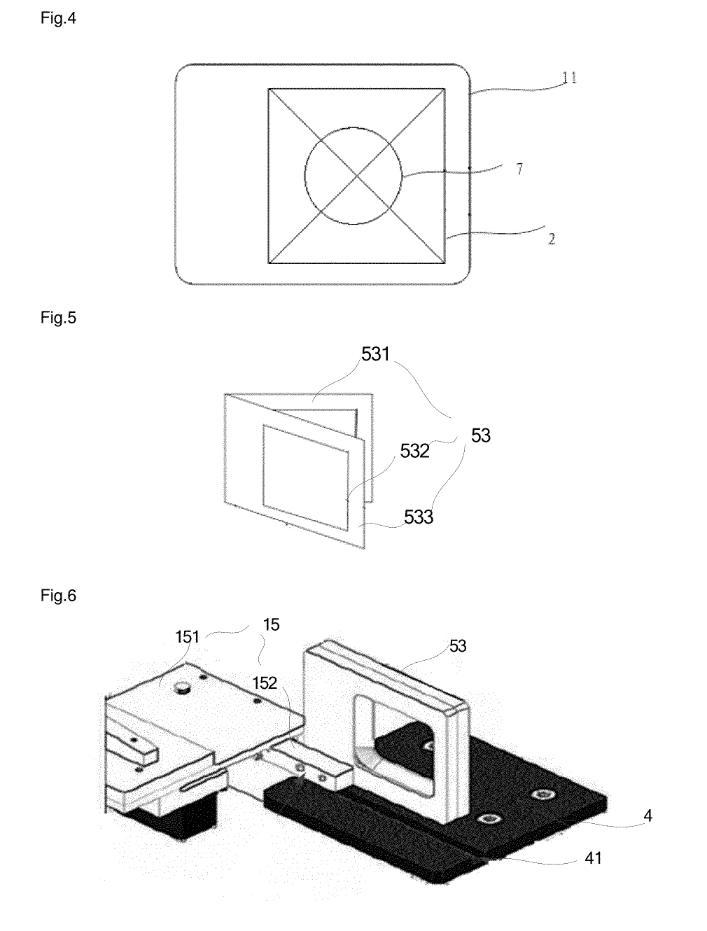

Further in some embodiments, to obtain a more accurate cornea positioning image, the center position of the camera is disposed on a perpendicular center line of the positioning screen 11. Referring to FIG. 4, exemplarily, a same cross template 2 is able to be configured to calibrate whether the center position of the image recognition system 7, i.e. the camera, and the center position of the positioning screen 11 are disposed by directly facing each other, in some embodiments, the cross template 2 is printed with an acetate ink.

In order to facilitate the corneal package body moving in a horizontal direction and a vertical direction, the mechanical arm assembly 5 comprises a horizontally moving assembly 52, a vertically moving assembly 51 and a clamping assembly 53. The horizontally moving assembly 52 is able to drive the vertically moving assembly 51 and the clamping assembly 53 to reciprocate in a horizontal direction parallel to the surface of the positioning screen 11. The vertically moving assembly 51 is able to drive the clamping assembly 53 to reciprocate in the vertical direction parallel to the surface of the positioning screen 11.

The specific structures of the horizontally moving component 52, the vertically moving component 51 and the clamping component 53 are not limited in embodiments of the present disclosure. Further and in some embodiments, the horizontally moving assembly 52 comprises a first driving member (not shown in a Fig), a horizontal guide rail 521, a horizontal lead screw disposed in the horizontal guide rail 521, and a first nut 522 matching the horizontal lead screw. The first nut 522 is slidably connected with the horizontal guide rail 521. The first driving member is able to drive the horizontal lead screw to rotate. The vertically moving assembly 51 is fixedly connected with the first nut 522.

The vertically moving assembly 51 comprises a second driving member, a vertical guide rail, a vertical lead screw disposed in the vertical guide rail, and a second nut matching the vertical lead screw. The second nut is slidably connected with the vertical guide rail. The second driving member may drive the vertical lead screw to rotate.

In embodiments of the present disclosure, the first driving member and the second driving member are not limited. For example, in order to enable the horizontally moving component 52 to drive the vertically moving component 51 and the clamping component 53 to reciprocate in the horizontal direction, in some embodiments, a motor is able to be adopted for the first driving member and the second driving member in embodiments of the present invention disclosure. The first nut 522 slides in the horizontal guide rail because of the first driving member driving the horizontal lead screw to rotate, and drives the vertically moving assembly 51 and the corneal package body to move in the horizontal direction. The second nut is able to drive the corneal package body to move up and down, when sliding along the vertical guide rail. Therefore, the corneal package body is able to move horizontally under the action of the horizontally moving assembly 52, and is able to move up and down under the action of the vertically moving assembly 51, so the movement of the corneal package body is achieved.

Exemplarily, in order to enable manual adjustment of the mechanical arm assembly 5, in some embodiments, a rotary button is disposed at one end of the horizontal guide rail 521. When the rotary button is rotated, the horizontal lead screw is able to be driven to rotate, making the vertically moving assembly 51 and the clamping assembly 53 move in the horizontal direction. Therefore, when the mechanical arm assembly 5 needs to be fine-tuned or fails, the mechanical arm assembly 5 is able to drive the vertically moving assembly 51 and the clamping assembly 53 to move in the horizontal direction by manually rotating the rotary button.

It should be noted that the mechanical arm assembly 5, the image recognition system 7, the background assembly 1 and the computing positioning system are able to be placed on a machine shelf 3 as shown in FIG. 1 in an actual operation. The machine shelf 3 is provided thereon with a stop button 10 which is configured to control the operating state of the whole positioning system. When the positioning system fails, the positioning system is controlled to be in a stopped state. A button 11 arranged on the machine shelf 3 is configured to control the mechanical arm assembly to be returned to an initial position.

Referring to FIGS. 5 and 6, in order to enable the mechanical arm assembly 5 to clamp tightly the corneal package body so as to drive the corneal package body to move in the horizontal direction or the vertical direction, in some embodiments, the clamping assembly 53 comprises a support component, a first clamping component 531, a second clamping component 533, a resetting component and a driving member 15.

A positioning window is disposed at each of the central portions of the first clamping component 531 and the second clamping component 533. The first clamping component 531 is fixed on the support component. The first clamping component 531 and the second clamping component 533 are oppositely disposed, and the first clamping component 531 and the second clamping component 533 are connected via the resetting component therebetween.

In order to enable the driving component 15 to drive the clamping assembly 53 to clamp the corneal package body, in some embodiments, the driving assembly component 15 comprises a motor 151. An output shaft of the motor 151 is connected with a swinging arm 152. One end of the swinging arm 152, away from the output shaft of the motor 151, abuts against the second clamping component 533. When the motor 151 is energized, the output shaft of the motor 151 drives the swinging arm 152 to swing toward the second clamping component 533. The swinging arm 152 is able to push the second clamping component 533 to rotate toward the first clamping component 531 until the first clamping component 531 touches the second clamping component 533 to further facilitate the clamping assembly 53 being in the clamped state. When the output shaft of the motor stops driving the swinging arm 152 to swing, the second clamping component 533 rotates away from the first clamping component 531 under action of the resetting component to further facilitate the clamping assembly 53 being in an open state.

It should be noted that, in the normal state, that is, the clamping assembly 53 is not in a working state, the clamping component 53 is in the open state as shown in FIG. 5, the resetting component positioned between the first clamping component 531 and the second clamping component 533 is in an initial state without being subjected to an external force. In the working state, the swinging arm 152 is driven by the motor 151 to apply an external force to the second clamping component 533, so that the second clamping component 533 is pressed by the external force which is exerted by the swinging arm 152 to rotate toward the first clamping component 531. Therefore, the resetting component positioned between the first clamping component 531 and the second clamping component 533 is deformed under the axial pressure exerted by the second clamping component 533 and the first clamping component 531. Further, the first clamping component 533 and the second clamping component 533 are facilitated being in the clamped state. When the external force applied to the second clamping component 533 disappears, that is, the motor 151 stops working and driving the swinging arm 152 to apply the external force to the second clamping component 533, the first clamping component 531 and the second clamping component 533 are restored back to the normal state under the action of the resetting component.

In order to facilitate the driving component 15 driving the swinging arm 152 to apply an external force to the second clamping component 533, in some embodiments, the driving component 15 in embodiments of the present disclosure is driven by a motor (not shown in a FIG.). When the motor 151 is energized, the motor 151 drives the driving component 15 to rotate, and the driving component 15 drives the swinging arm 152 to apply the external force to the second clamping component 533. When the motor 151 is powered off, the driving component 15 stops rotating, the external force applied to the second clamping component 533 disappears, and the clamping assembly 53 is restored back to the normal state under the action of the resetting component.

It should be noted that, in the practical use, the driving component 15 in embodiments of the present disclosure is also able to apply an external force to the first clamping component 531 through the swinging arm 152, making the first clamping component 531 rotate toward the second clamping component 533 under the action of external force. Embodiment of the present disclosure does not limit this, as long as the corneal package body is able to be clamped tightly, by the first clamping component 531 and the second clamping component 533 in the working state.

Exemplarily, when the corneal package body needs to be clamped tightly, the corneal package body containing a cornea to-be-positioned is placed and maintained between the first clamping component 531 and the second clamping component 533 when the clamping assembly 53 is in the normal state. Energizing the motor 151, the motor drives the driving component 151 to rotate so as to drive the swinging arm 152 to rotate, so that one end of the swinging arm 152, far from the output shaft of the motor 151, presses the second clamping component 533 to rotate toward the first clamping component 531, and therefore, the first clamping component 531 and the second clamping component 533 press the resetting component under the action of the external force to clamp tightly the corneal package body containing the cornea to-be-positioned.

It should be noted that, in order to obtain a better cornea positioning image, when the clamping assembly 53 clamps the corneal package body between the image recognition system 7 and the background assembly 1, the center position of the positioning window 532 is directly opposite to the center position of the positioning screen 11.

The specific shapes of the first clamping component 531 and the second clamping component 533 in embodiments of the present disclosure are not limited, exemplarily, are able to be designed according to the shape of the corneal package body. For example, the first clamping component 531 and the second clamping component 533 are able to be designed in a sheet shape, as long as the first clamping component 531 and the second clamping component 533 are able to clamp tightly the corneal package body.

In embodiments of the present disclosure, the resetting component is not limited. For example, an elastic component is able to be adopted for the resetting component. The coefficient of elasticity of the elastic component is not specifically limited in embodiments of the present disclosure, as long as the elastic component enables a maximum opening distance between the first clamping component 531 and the second clamping component 533 to be greater than or equal to the thickness of the corneal package body when the first clamping component 531 and the second clamping component 533 are in the open state, or as long as when the motor 151 drives the swinging arm 152 to rotate to press the second clamping component 533 to rotate toward the first clamping component 531, the elastic component positioned between the first clamping component 531 and the second clamping component 533, is able to facilitate the first clamping component 531 and the second clamping component 533 being able to clamp tightly the corneal package body, when being pressed under the external force to be deformed. In some embodiments, the elastic component is a spring.

Referring to FIG. 6, in order to conveniently move the corneal package body to be under the clamping component, embodiments of the present disclosure further comprises a sample stage 4. The sample stage 4 is configured to place the corneal package body (not shown in the FIG). The specific structure of the sample stage 4 is not limited. The sample stage 4 is disposed below the initial position of the clamping assembly 53. The sample stage 4 is provided thereon with a limiting groove 41. The limiting groove 41 is positioned directly under a gap between the first clamping component 531 and the second clamping component 533 when the first clamping component 531 and the second clamping component 533 are in the clamped state.

Exemplarily, in the practical use, the corneal package body is moved along the limiting groove 41 of the sample stage 4 to be below the initial position of the clamping component 53. The width of the limiting groove 41 is the same as the thickness of the corneal package body. The corneal package body is able to be manually moved or moved by the main control system, along the limiting groove 41, to be below the initial position of the clamping assembly 53, which is not limited in embodiments of the present disclosure.

The initial position is a preset initialization state of the clamping component 53 on the mechanical arm assembly 5. The preset initialization state is able to be preset according to the system, or to be any position of the clamping component 53 on the mechanical arm assembly 5 after a previous cornea positioning is completed.

The computing positioning system is computer-centric, and uses a mathematical technique method to correspondingly process an image obtained by one front end of the system according to a specific purpose. On a digital image processing system, including various input, output and display devices, a processing to run and to meet various requirements, is implemented. The process is controlled by a program which is programmed based on specific physical and mathematical models after a continuous analog image is converted into a discrete digital image. The computing positioning system may be implemented by using the prior art, which is not limited in this embodiment of the present disclosure.

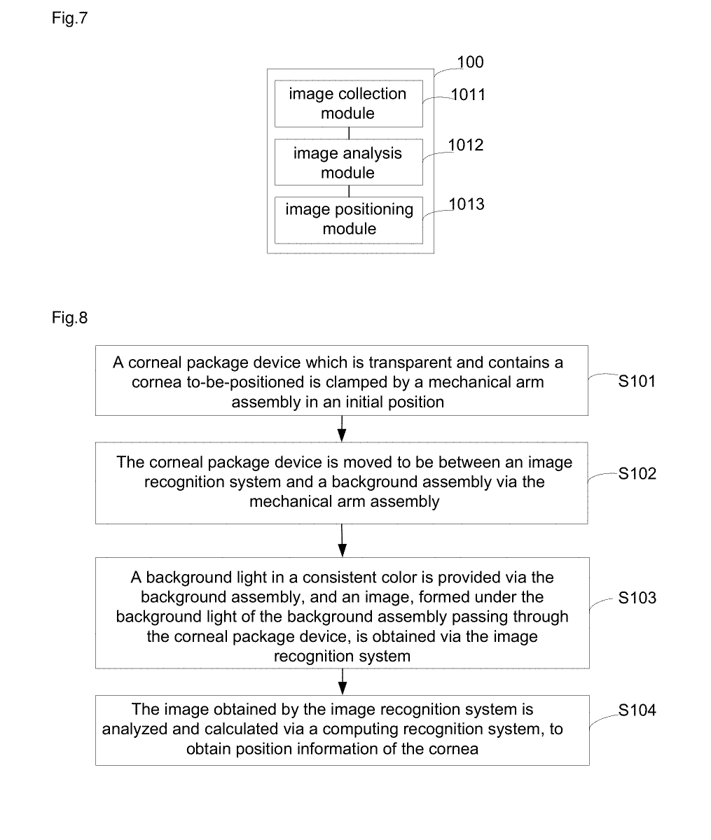

Referring to FIG. 7, exemplarily, the computing positioning system 100 in embodiments of the present disclosure comprises:

an image collection module 1011, configured to collect parameter information of each pixel point in the image obtained by the image recognition system;

an image analysis module 1012, configured to obtain, according to a set parameter threshold range, a pixel point in the image, of which the parameter information meets the threshold range; and

an image positioning module 1013, configured to determine the position of the cornea being the coordinate of the pixel point that meets the threshold range.

Exemplarily, the cornea positioning is taken as an example, an image formed on the positioning screen through the background light, obtained by the camera, is collected by the image collection module, and is converted into a 24-bit RGB image. That is, the parameter information of each pixel point in the image is obtained. The parameter information at least comprises the width Bw, the height Bh, the image resolution Bf and the coordinate, taking a pixel as a unit, of the image. In some embodiments, the present disclosure uses the coordinate of each pixel in the image as an example for description. The coordinate position of each image pixel point in the coordinate system is obtained by the collection module. By comparing the coordinate corresponding to each pixel point in the coordinate system with the preset coordinate position, the coordinate position of each pixel point that meets the preset threshold range is selected. And with the coordinate of the pixel point that meets the requirement, the cornea is positioned.

The preset coordinate position may be preset by the computing positioning system, or may be obtained according to the correlation between the coordinate of each image pixel point in the coordinate system.

Exemplarily, to obtain, according to the set parameter threshold range, the pixel point in the image, of which the parameter information meets the threshold range according to the set parameter threshold range, further comprises:

classifying the pixel points of the image according to the coordinate position corresponding to each pixel point in the coordinate system, classifying the pixel points of which the coordinate positions meet certain constraints into one class, for example, classifying the pixel points that meet a linear constraint and are near an offset of the linear constraint into one class when some pixel points in the coordinate system meet the certain linear constraint, selecting the corresponding coordinate positions of the pixel points in the coordinate system that meet the linear constraint and are near the offset of the linear constraint, and positioning the cornea with the coordinates of the pixel points that meet the requirement,

wherein it should be noted that the linear constraint may be preset by the system, or obtained according to the decentralization rule of each pixel point in the coordinate system;

or drawing a circular area which is centered at a certain optimal pixel point in the coordinate system and with a preset threshold as the offset, classifying the pixel points falling within the circular area into one class, selecting the coordinate positions of the pixel points falling within the circular area, and positioning the cornea with the coordinates of the pixel points that meet the requirement,

Wherein it should be noted that the optimal pixel point may be a preset pixel point, or may be a selected pixel point that meets the requirement most according to the corresponding position relationship of each pixel point in the coordinate system, for the offset, the present disclosure is not limited thereto, in the practical use, and a user may perform presetting via the system according to his needs or may perform selection according to the distribution of each pixel point in the coordinate system.

In some embodiments of the present disclosure, in order to perform automatic control of each operation during the cornea positioning and reduce manual intervention during the cornea positioning, the cornea positioning system according to embodiments of the present disclosure further comprises the main control system and the mechanical control system connected with the main control system. The mechanical control system is electrically connected with the mechanical arm assembly and configured to control the mechanical arm assembly to move the corneal package body which is transparent and contains a cornea to-be-positioned to be between the image recognition system and the background assembly.

The main control system is electrically connected with the image recognition system and the computing positioning system respectively, and configured to control the image recognition system and the computing positioning system to obtain the image, formed under the action of the background light, of the corneal package body when the mechanical arm assembly moves the corneal package body to be between the image recognition system and the background assembly, and to analyze and calculate the image obtained by the image recognition system to obtain the position information of the cornea.

It should be noted that in, in embodiments of the present disclosure during a practical use process, according to a control signal sent by the main control system, the mechanical control system controls the mechanical assembly 5 to move the corneal package body to be between the image recognition system 7 and the background assembly 1. When the corneal package body is moved to be between the image recognition system 7 and the background assembly 1, the mechanical control system feeds back to the main control system a signal of the corneal package body being moved to be between the image recognition system 7 and the background assembly 1. At this point, the background assembly 1 is able to be controlled by the main control system to provide the background light, or the background assembly 1 is able to be manually facilitated to provide the background light, which is not limited in embodiments of the present disclosure.

In some embodiments, in embodiments of the present disclosure, a background light in a consistent color is able to be provided via the background assembly, so that the background light passes through the corneal package body clamped by the clamping component 53 to form an image on the positioning screen 11.

In summary, during the entire operation process of the cornea positioning system, each operation process is automatically controlled by the mechanical control system, thereby reducing manual intervention in the cornea positioning system and being capable of improving the accuracy of the cornea positioning.

An embedded Arcus controller is used for the mechanical control system. The Arcus controller has eight digital input ports and eight digital output ports. The input ports are configured to obtain an instruction from the main control system to move the corneal package body to be between the background assembly 1 and the image recognition system 7, and when the image recognition system 7 detects that the center position coordinate of the positioning window 532 does not match the preset center coordinate, that is, there is a deviation, the center position of the positioning window 532 is adjusted according to the deviation via the Arcus controller. The output ports are configured to feed back a completion state to the main control system according to an instruction sent by the main control system.

The main control system 14 is able to comprise one or more microprocessors 13, a memory, a user interface, a network interface and a communication bus.

The communication bus is configured to control the communication among the various components of the cornea positioning system. The user interface is configured to plug in an external apparatus such as a touch screen, a mouse, a keyboard and the like to receive information input by a user. The network interface is configured to facilitate the controller communicating with the outside, and the network interface mainly comprises a wired interface and a wireless interface.

The memory is able to be configured to store a software program, a module and a database, for example, a program instruction/a module corresponding to a cornea positioning method, a program instruction/a module corresponding to a fact that the mechanical control system controls the mechanical arm assembly to drive the corneal package body to move in the horizontal direction or in the vertical direction, a program instruction/a module corresponding to a fact that the background assembly provides the background light, or a program instruction/a module that the computing positioning system analyzes and calculates the image obtained by the image recognition system, in embodiments of the present disclosure. The memory is able to comprise a high-speed random access memory, and is also to comprise a non-volatile memory, such as one or more magnetic storage devices, a flash memory, or other non-volatile solid-state memory. In some examples, the memory is further to comprise a memory remotely disposed relative to the microprocessor. These remote memories are able to be connected to the control apparatus via a network. The examples of the above network comprise, but are not limited to, the Internet, a corporate intranet, a local area network, a mobile communication network and a combination thereof.

The microprocessor 13 executes various functional applications and data processing by running the software program instructions and the modules stored in the memory, for example, the processor realizes the cornea positioning process quickly and accurately by calling a cornea positioning application in the memory, and realizes the process of driving the corneal package body to move in the horizontal direction or in the vertical direction by calling the program instruction/module in the memory corresponding to the fact that the mechanical control system controls the mechanical assembly to move.

In some embodiments, an Arduino pro is adopted for the microprocessor of the present disclosure. An output of the Arduino pro is connected to a digital input port of the Arcus controller. An input of the Arduino pro is connected to the main control system.

In summary, the main control system may realize automated control in each operation step of the control process, only by inputting a set operation program to the main control system 14. In a practical use, the microprocessor 13 calls an application program stored in the main control system 14 to complete the entire operation.

Referring to FIG. 8, in another aspect, embodiments of the present disclosure further provides a cornea positioning method, which is applied to the cornea positioning system described above in FIG. 1 and FIG. 2, comprising the following steps.

S101, a corneal package body containing a cornea to-be-positioned is clamped by a mechanical arm assembly 5 in an initial position.

S102, the corneal package body is moved to be between an image recognition system 7 and a background assembly 1 via the mechanical arm assembly.

S103, a background light is provided via the background assembly 1, and an image, formed under action of the background light, of the corneal package body is obtained via the image recognition system. and

S104, the image obtained by the image recognition system is analyzed and calculated via a computing positioning system, to obtain position information of the cornea.

According to the cornea positioning method provided by embodiments of the present disclosure, when a cornea in a corneal package body transparent and needs to be positioned, the corneal package body which is transparent and contains the cornea to-be-positioned is clamped by the mechanical arm assembly in the initial position, then the corneal package body is moved to be between the image recognition system and the background assembly via the mechanical arm assembly, then a background light in a consistent color is provided by the background assembly, an image, formed under the background light of the background assembly passing through the corneal package body clamped by the mechanical arm assembly, is obtained via the image recognition system, and finally, the image obtained by the image recognition system is analyzed and calculated via the computer positioning system to obtain the position information of the cornea. This cornea positioning method may not only improve accuracy of the cornea positioning but also reduce the labor of workers.

The mechanical arm assembly comprises a horizontally moving assembly 52, a vertically moving assembly 51 and a clamping assembly 53. The horizontally moving assembly 52 is able to drive the vertically moving assembly 51 and the clamping assembly 53 to reciprocate in a horizontal direction parallel to a surface of a positioning screen 11. The vertically moving assembly 51 is able to drive the clamping assembly 53 to reciprocate in a vertical direction parallel to the surface of the positioning screen 11. The horizontally moving assembly 52 comprises a first driving member, a horizontal guide rail, a horizontal lead screw disposed in the horizontal guide rail, and a first nut matching the horizontal lead screw. The first nut is slidably connected with the horizontal guide rail. The first driving member may drive the horizontal lead screw to rotate. The vertically moving assembly is fixedly connected with the first nut. The vertically moving assembly comprises a second driving member, a vertical guide rail, a vertical lead screw disposed in the vertical guide rail, and a second nut matching the vertical lead screw, the second nut is slidably connected with the vertical guide rail. The second driving member is able to drive the vertical lead screw to rotate.

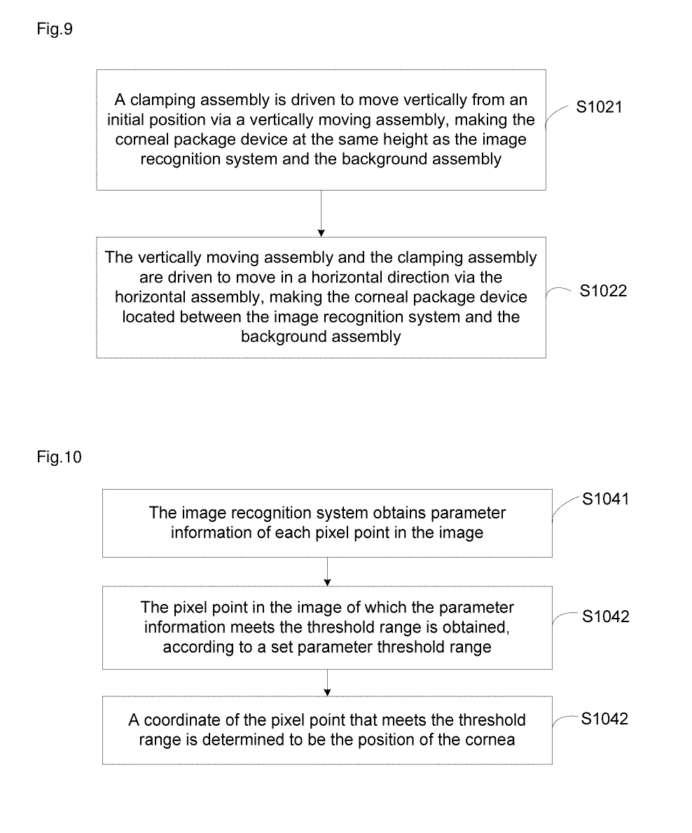

Referring to FIG. 9, exemplarily, step of S102 is able to be specifically realized by the following steps:

S1021, the second driving member is switched on, and drives the vertical lead screw to rotate to drive the clamping assembly to move vertically from the initial position in a direction parallel to the surface of the positioning screen, and the second driving member is switched off when the corneal package body is at the same height as the image recognition system and the background assembly.

S1022, the first driving member is switched on, and drives the horizontal lead screw to be rotate to drive the vertical moving assembly and the clamping assembly to move in the horizontal direction parallel to the surface of the positioning screen, and the second driving member is switched off when the corneal package body is located between the image recognition system and the background assembly.

Further, in order to facilitate the mechanical arm assembly driving the corneal package body to move in the horizontal direction or the vertical direction, in some embodiments, the clamping assembly comprises a support component, a first clamping component, a second clamping component, a resetting component, and a driving component.

A positioning window is disposed at each of the central portions of the first clamping component and the second clamping component. The first clamping component is fixed on the support component. The first clamping component and the second clamping component are oppositely disposed, and the first clamping component and the second clamping component are connected via the resetting component therebetween. The driving component comprises a motor. An output shaft of the motor is connected with a swinging arm. One end of the swinging arm, away from the output shaft of the motor, abuts against the second clamping component.

Exemplarily, the step of S101 is able to be realized by the following steps.

S1011, the corneal package body containing the cornea to-be-positioned is placed and maintained between the first clamping component and the second clamping component, when the motor is switched off, and the second clamping component and the first clamping component are in an open state under action of the resetting component.

An external force applied to the second clamping component and the first clamping component disappears when the motor is switched off. The second clamping component and the first clamping component are restored back to the initial position under the action of the resetting component, and at this point, the first clamping component and the second clamping component are in the open state depending on an elastic force of the resetting component itself. When the first clamping component and the second clamping component are in the open state, the distance between the first clamping component and the second clamping component shall be greater than or equal to the thickness of the corneal package body.

S1012, the motor is switched on, the output shaft of the motor drives the swinging arm to swing toward the second clamping component, and the swinging arm pushes the second clamping component to rotate toward the first clamping component until the first clamping component touches the second clamping component.

An external force is applied to the swinging arm by switching on the motor, the swinging arm is driven by the motor to rotate, so the swinging arm is driven to press the second clamping component to rotate toward the first clamping component. Therefore, the resetting component between the first clamping component and the second clamping component is pressed so that the first clamping component and the second clamping component clamp tightly the corneal package body which is transparent and contains the cornea to-be-positioned.

Further, the corneal package body between the image recognition system and the background assembly means that the center position of the positioning window of the clamping component is directly facing the center position of the positioning screen. In order to obtain better accuracy of the cornea positioning, the size and the position of the positioning window are not limited, as long as when the corneal package body is located between the image recognition system and the background assembly, the center position of the positioning window 532 of the clamping assembly 53 is directly facing the center position of the positioning screen by the horizontally moving assembly 52 and the vertical moving assembly 51.

That is, referring to FIG. 10, exemplarily, the step of S104 is specifically to be realized by the following steps.

S1041, the image recognition system obtains parameter information of each pixel point in the image.

S1042, the pixel point in the image of which the parameter information meets the threshold range is obtained. according to a set parameter threshold range. and

S1043, a coordinate of the pixel point that meets the threshold range is determined to be the position of the cornea.

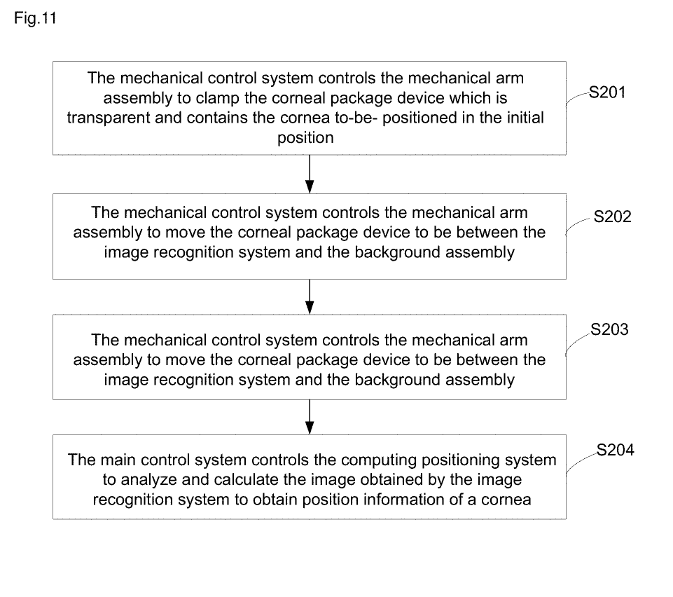

Referring to FIG. 11, some embodiments of the present disclosure provides a cornea positioning method, the corneal positioning system applied to the cornea positioning method comprises the mechanical arm assembly, the image recognition system, the background assembly disposed opposite to the image recognition system, the computing positioning system connected with the image recognition system, a main control system, and a mechanical control system connected with the main control system.

The mechanical control system is electrically connected with the mechanical arm assembly and configured to control the mechanical arm assembly to move a corneal package body containing a cornea to be between the image recognition system and the background assembly.

The main control system is electrically connected with the image recognition system and the computing positioning system respectively, is configured to control the image recognition system to obtain an image, formed under the action of a background light, of the corneal package body when the mechanical arm assembly moves the corneal package body to be between the image recognition system and the background assembly, and is configured to control the computing positioning system to analyze and calculate the image obtained by the image recognition system to obtain the position information of the cornea. This method comprises:

S201, the mechanical control system controls the mechanical arm assembly to clamp the corneal package body containing the cornea to-be-positioned in the initial position;

S202, the mechanical control system controls the mechanical arm assembly to move the corneal package body to be between the image recognition system and the background assembly;

S203, the main control system, controls the background assembly to provide the background light, and controls the image recognition system to obtain the image, formed under the action of the background light, of the corneal package body; and

S204, the main control system controls the computing positioning system to analyze and calculate the image obtained by the image recognition system to obtain position information of a cornea.

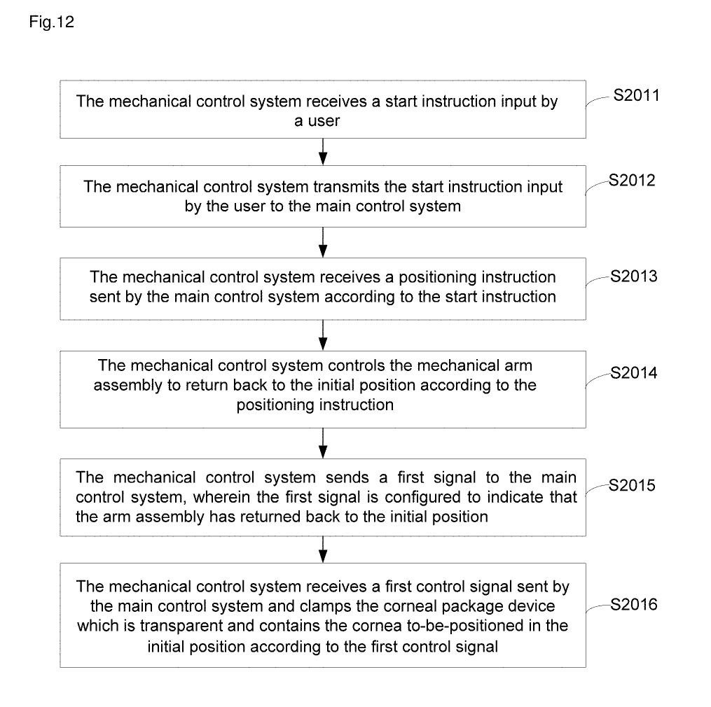

Referring to FIG. 12, exemplarily, the step of S201 may specifically comprises the following steps.

S2011, the mechanical control system receives a start instruction input by a user.

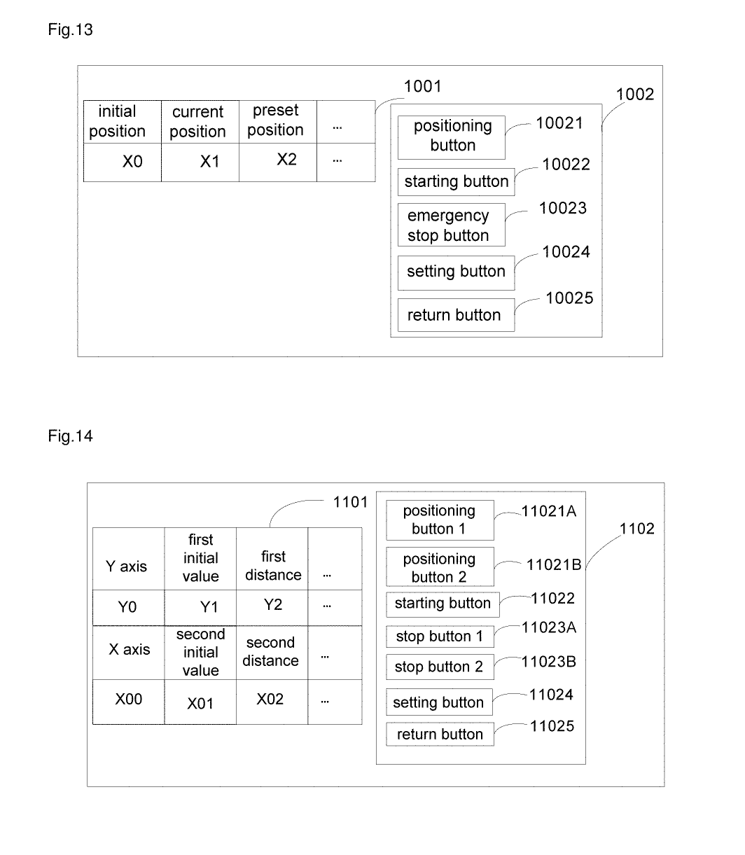

Specifically, the user is able to input an instruction, for starting work of the arm assembly, in an operation interface of the mechanical control system. For example, a running button disposed on the operation interface of the mechanical arm control system is switched on to send an instruction to the mechanical control system, to start the work of the mechanical arm assembly. The operation interface of the mechanical control system may refer to the operation interface shown in FIG. 13. A left portion 1001 of the operation interface is configured to display running state information of the mechanical arm assembly as a whole, such as the initial position, a current position, a preset position, a distance between the current position and the initial position, etc. of the mechanical arm assembly. A right portion 1002 of the interface is configured to control running state of the mechanical arm control system, for example, the running button 10021 is configured to input a start instruction for starting the work of the mechanical arm assembly, a positioning button 10022 is configured to move the mechanical arm assembly to the preset position via a limiting sensor, a starting button 10023 is configured to facilitate the mechanical arm assembly return back to a starting position during operation, an emergency stop button 10024 is configured to stop a movement state of the mechanical arm assembly and that of the mechanical control system, a setting button 10025 is configured to set a running parameter for the mechanical arm assembly to move to the preset position, and a return button 10026 is configured to exit from the operation interface of the mechanical control system.

It should be noted that FIG. 13 is merely an exemplary diagram of an operation interface of a mechanical control system, of course, there may be other possible operation interfaces, which are not specifically limited in embodiments of the present disclosure.

S2012, the mechanical control system transmits the start instruction input by the user to the main control system.

S2013, the mechanical control system receives a positioning instruction sent by the main control system according to the start instruction.

S2014, the mechanical control system controls the mechanical arm assembly to return back to the initial position according to the positioning instruction.

S2015, the mechanical control system sends a first signal to the main control system, wherein the first signal is configured to indicate that the arm assembly has returned back to the initial position.

S2016, the mechanical control system receives a first control signal sent by the main control system and clamps the corneal package body containing the cornea to-be-positioned in the initial position according to the first control signal.

Further, in order to facilitate the controller to control the corneal package body to move in the horizontal direction and the vertical direction, the mechanical arm assembly 5 comprises a horizontally moving component 52, a vertically moving component 51 and a clamping component 53. The horizontally moving component 52 is able to drive the vertically moving component 51 and the clamping assembly 53 to reciprocate in a horizontal direction parallel to the surface of the positioning screen 11. The vertically moving assembly 51 may drive the clamping assembly 53 to reciprocate in a vertical direction parallel to the surface of the positioning screen 11. The horizontally moving assembly 52 comprises a first driving member, a horizontal guide rail, a horizontal lead screw disposed in the horizontal guide rail, and a first nut matching the horizontal lead screw. The first nut is slidably connected with the horizontal guide rail. The first driving member is able to drive the horizontal lead screw to rotate. The vertically moving assembly is fixedly connected with the first nut. The vertically moving assembly 51 comprises a second driving member, a vertical guide rail, a vertical lead screw disposed in the vertical guide rail, and a second nut matching the vertical lead screw. The second nut is slidably connected with the vertical guide rail. The second driving member is able to drive the vertical lead screw to rotate.

The initial position comprises a coordinate formed by a first initial value of the second nut at the vertical guide rail and a second initial value of the first nut 522 at the horizontal guide rail.

Exemplarily, the step of S2014 is able to be specifically realized by the following steps.

S20141, the mechanical control system detects a current value of the first nut on the horizontal guide rail and a current value of the second nut on the vertical guide rail.

S20142, the mechanical control system controls the first driving member to be switched on to drive the vertically moving assembly and the clamping assembly to move along the horizontal guide rail to be at a second initial value when the current value of the second nut on the vertical guide rail is the first initial value and the current value of the first nut on the horizontal guide rail is not the second initial value.

S20143, the mechanical control system controls the second nut to drive the clamping assembly to move along the vertical guide rail to be at the first initial value when the current value of the second nut on the vertical guide rail is not the first initial value and the current value of the first nut on the horizontal guide rail is the second initial value.

S20144, the mechanical control system controls the first nut to drive the clamping assembly to move along the horizontal guide rail to be at the second initial value, and controls the first nut to drive the clamping assembly to move along the vertical guide rail to be at the first initial value when the current value of the second nut on the vertical guide rail is not the first initial value, and the current value of the first nut on the horizontal guide rail is not the second initial value.

The first initial value is preset by the main control system, or is obtained by the main control system according to the current value of the second nut on the vertical guide rail, the current value of the first nut on the horizontal guide rail, and the position between the first initial value and the second initial value.

In order to facilitate the mechanical control system controlling the mechanical arm assembly to clamp tightly the corneal package body, the clamping assembly comprises a support component, a first clamping component, a second clamping component, a resetting component, and a driving component.

A positioning window is disposed at each of central portions of the first clamping component and the second clamping component. The first clamping component is fixed on the support component. The first clamping component and the second clamping component are oppositely disposed. The first clamping component and the second clamping component are connected via the resetting component therebetween. The driving component comprises a motor. An output shaft of the motor is connected with a swinging arm. One end of the swinging arm, away from the output shaft of the motor, abuts against the second clamping component. When the output shaft of the motor drives the swinging arm to swing toward the second clamping component, the swinging arm is able to push the second clamping component to rotate toward the first clamping component until the first clamping component touches the second clamping component. When the output shaft of the motor stops driving the swinging arm to swing, the second clamping component rotates away from the first clamping component under the action of the resetting component.

For example, the mechanical control system controlling the mechanical arm assembly to clamp the corneal package body transparent and containing the cornea to-be-positioned in the initial position, comprises:

the mechanical control system controls the motor to be switched off, and the second clamping component and the first clamping component are in the open state under the action of the resetting component;

the corneal package body containing the cornea is placed and maintained between the first clamping component and the second clamping component;

the mechanical control system switch on the motor at a second time after a preset period, wherein the output shaft of the motor drives the swinging arm to swing toward the second clamping component, and the swinging arm pushes the second clamping component to rotate toward the first clamping component until the first clamping component touches the second clamping component.

The preset time is preset by the system, or is determined by the main control system according to a time from when the first clamping component and the second clamping component are in the open state to when they clamp tightly the corneal package body containing the cornea. The second time is preset by the system, or is determined by the main control system according to a time how long an elastic component takes to make the first clamping component and the second clamping component be in the open state. When the clamping assembly is in the open state, the distance between the first clamping component and the second clamping component shall be greater than or equal to the thickness of the corneal package body, that is, the second time is calculated and obtained according to the distance between the first clamping component and the second clamping component, and the speed of the elastic component to make the first clamping component and the second clamping component to be in the open state.

Exemplarily, the mechanical control system controlling the mechanical arm assembly to move the corneal package body to be between the image recognition system and the background assembly, comprises:

the mechanical control system controls the second driving member to be switched on to drive the clamping assembly to slide downward a first distance along the vertical guide rail from the first initial value by;

the mechanical control system controls the second driving member to be switched off after the vertical guide rail slides downward the first distance making the corneal package body be at the same height as the image recognition system and the background assembly;

the mechanical control system controls the first driving member to be switched on to drive the vertically moving assembly and the clamping assembly to move a second distance along the horizontal guide rail from the second initial value in the first direction, wherein the first direction is a direction of the horizontal guide rail toward a position between the image recognition system and a background assembly; and

the mechanical control system controls the first driving member to be switched off after the first nut moves the second distance along the horizontal guide rail in the first direction, making the corneal package body be located between the image recognition system and the background assembly.

Referring to FIG. 14, FIG. 14 is a diagram of an operation interface of the mechanical control system according to embodiments of the present disclosure, A left-side operation interface 1101 is configured to display position information of the mechanical arm assembly 5 in a Y axis (the vertical guide rail) and an X axis (the horizontal guide rail), for example, a first initial position, a second initial position, the first distance and the second distance. A right-side operation interface 1102 is configured to control the parameters of the mechanical arm assembly 5, for example, a positioning button 11021A is configured to facilitate the second nut driving the clamping assembly 53 to slide downward the first distance from the first initial value along the vertical guide rail via the limiting sensor, a positioning button 11021B is configured to facilitate the first nut driving the vertically moving assembly 51 and the clamping assembly 53 to move the second distance along the horizontal guide rail from the second initial position in the first direction via the limiting sensor, a starting button 110022 is configured to facilitate the mechanical arm assembly 5 returning back to the first initial position or the second initial position during operation, a stop button 110023A is configured to facilitate the first nut stopping moving on the vertical guide rail when the clamping assembly 53 has moved to the first distance along the vertical guide rail, a stop button 11023B is configured to facilitate the first nut stopping moving on the horizontal guide rail after the clamping assembly 53 has moved to the second distance in a horizontal guide rail, a setting button 11024 is configured to set the parameters of the preset positions of the mechanical arm assembly on the vertical guide rail and the horizontal guide rail, and a return button 11025 is configured to exit from the operating interface of the mechanical control system.

It should be noted that FIG. 14 is merely an exemplary diagram of an operation interface of the mechanical control system, of course, there may be other possible operation interfaces, which are not specifically limited in this embodiment of the present disclosure.

The first distance is preset by the main control system according to the difference in height between the image recognition system 7 together with the background assembly 1 and the first initial position of the second nut in the vertical guide rail. The second distance is preset by the main control system according to the difference of positions, in the horizontal direction, between the image recognition system 7 together with the background assembly 1 and the second initial position of the first nut in the horizontal guide rail.

In order to make the positioning effect more accurate, after the mechanical control system controls the mechanical arm assembly 5 to move the corneal package body to be between the image recognition system 7 and the background assembly 1, and before the main control system controls the computing positioning system to analyze and calculate the image obtained by the image recognition system 7 to obtain the position information of the cornea, the method further comprises:

that the main control system controls the image recognition system and the mechanical control system, making a center position of the positioning window of the clamping assembly located on the perpendicular center line of the positioning screen.

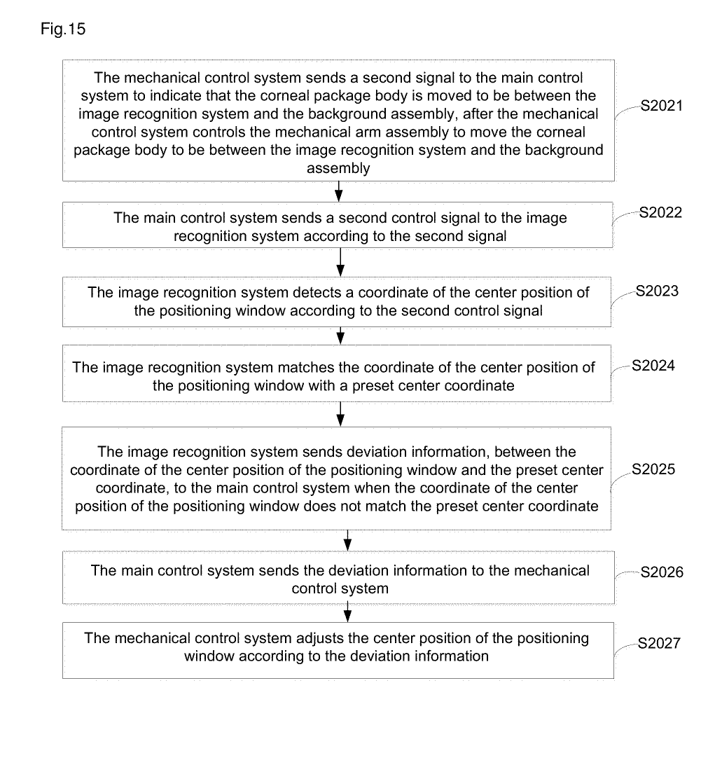

Referring to FIG. 15, exemplarily, the main control system controlling the image recognition system and the mechanical control system, making the center position of the positioning window of the clamping assembly located on the perpendicular center line of the positioning screen, comprises:

S2021, the mechanical control system sends a second signal to the main control system to indicate that the corneal package body is moved to be between the image recognition system and the background assembly, after the mechanical control system controls the mechanical arm assembly to move the corneal package body to be between the image recognition system and the background assembly;

S2022, the main control system sends a second control signal to the image recognition system according to the second signal; S2023, the image recognition system detects a coordinate of the center position of the positioning window according to the second control signal;

S2024, the image recognition system matches the coordinate of the center position of the positioning window with a preset center coordinate;

S2025, the image recognition system sends deviation information, between the coordinate of the center position of the positioning window and the preset center coordinate, to the main control system when the coordinate of the center position of the positioning window does not match the preset center coordinate;

S2026, the main control system sends the deviation information to the mechanical control system; and

S2027, the mechanical control system adjusts the center position of the positioning window according to the deviation information.

It should be noted that, when the mechanical arm assembly 5 moves the corneal package body to be between the image recognition system and the background assembly 1, the center position of the positioning window may be different from the preset center coordinate due to various factors, for example, the factor could be the difference caused by a deviation set by the system. Therefore, after obtaining the second signal, the main control system is able to check whether there is a difference between the coordinate of the center position of the positioning window 532 and the preset center coordinate, if yes, the mechanical control system adjusts the center position of the positioning window 532 to be consistent with the preset position according to the deviation information. This facilitates the center of the camera being directly opposite to the center position of the positioning window during following positioning processes, thereby enhancing the accuracy of the whole positioning system.

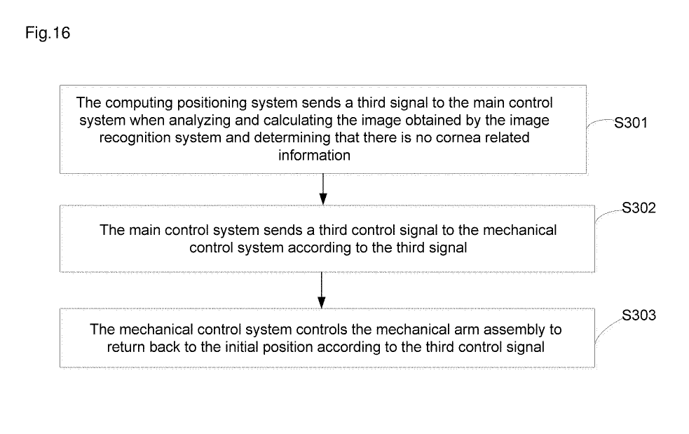

Referring to FIG. 16, exemplarily, embodiments of the present disclosure further comprise:

S301, the computing positioning system sends a third signal to the main control system when analyzing and calculating the image obtained by the image recognition system and determining that there is no cornea related information;

S302, the main control system sends a third control signal to the mechanical control system according to the third signal; and

S303, the mechanical control system controls the mechanical arm assembly to return back to the initial position according to the third control signal.

It should be noted that, during an actual operation, since there is no limitation on the cornea to-be-positioned, there may be no corneal information in the image formed via the background light during positioning. At this point, in order to improve the accuracy and efficiency of the positioning, the main control system is able to send the information, that no cornea is obtained, to the mechanical control system to control the mechanical control system to return the mechanical arm assembly to the initial position for the positioning of a next cornea.

According to a cornea positioning method provided by embodiments of the present disclosure, when a cornea in the corneal package body needs to be positioned, the mechanical control system first controls the mechanical arm assembly to clamp the corneal package body containing the cornea to-be-positioned in the initial position, then the mechanical control system controls the mechanical arm assembly to move the corneal package body to be between the image recognition system and the background assembly, then the main control system controls the background assembly to provide a background light in a consistent color and controls the image recognition system to obtain the image formed via the background light of the background assembly passing through the corneal package body clamped by the mechanical arm assembly, and finally, the main control system controls the computing positioning system to analyze and calculate the image obtained by the image recognition system to obtain the position information of the cornea. This cornea positioning method may not only improve the accuracy of the cornea positioning but also reduce the labor of workers.

The foregoing descriptions are merely specific embodiments of the present disclosure, but the protection scope of the present disclosure is not limited thereto. Any variation or replacement easily conceivable by those skilled in the art in the technical scope disclosed by the present disclosure, shall be within the protection scope of the present disclosure. Therefore, the protection scope of the present disclosure should be subject to the protection scope of the claims.

What is claimed is:

1. A cornea positioning system, comprising:
a mechanical arm assembly, an image recognition system, a background assembly disposed opposite to the image recognition system, and a computing positioning system connected to the image recognition system, wherein the mechanical arm assembly is configured to move a corneal package body containing a cornea to-be-positioned to be between the image recognition system and the background assembly;
the background assembly is configured to provide background light;
the image recognition system is configured to obtain an image, formed under action of the background light, of the corneal package body;
the computing positioning system is configured to analyze and calculate the image obtained by the image recognition system to obtain position information of the cornea.

2. The cornea positioning system according to claim 1, wherein the system further comprises a main control system and a mechanical control system,
wherein the mechanical control system is electrically connected with the mechanical arm assembly and configured to control the mechanical arm assembly to move the corneal package body containing the cornea to-be-positioned to be between the image recognition system and the background assembly;
the main control system is electrically connected with the image recognition system and the computing positioning system; is configured to control the image recognition system to obtain, when the mechanical arm assembly moves the corneal package body to be between the image recognition system and the background assembly, the image, formed under the action of the background light, of the corneal package body; and is configured to control the computing positioning system to analyze and calculate the image obtained by the image recognition system to obtain the position information of the cornea.

3. The cornea positioning system according to claim 1 wherein the image recognition system comprises a camera, and the background assembly comprises a positioning screen and a light source, wherein the light source is disposed behind the positioning screen relative to the image recognition system.

4. (canceled)

5. The cornea positioning system according to claim 1 wherein the mechanical arm assembly comprises a horizontally moving assembly, a vertically moving assembly and a clamping assembly, wherein the horizontally moving assembly is able to drive the vertically moving assembly and the clamping assembly to reciprocate in a horizontal direction parallel to a surface of the positioning screen, and the vertically moving assembly is able to drive the clamping assembly to reciprocate in a vertical direction parallel to the surface of the positioning screen.

6. The cornea positioning system according to claim 5, wherein the horizontally moving assembly comprises a first driving member, a horizontal guide rail, a horizontal lead screw disposed in the horizontal guide rail, and a first nut matching the horizontal lead screw, wherein the first nut is slidably connected with the horizontal guide rail, the first driving member is able to drive the horizontal lead screw to rotate, and the vertically moving assembly is fixedly connected with the first nut; and
the vertically moving assembly comprises a second driving member, a vertical guide rail, a vertical lead screw disposed in the vertical guide rail, and a second nut matching the vertical lead screw, wherein the second nut is slidably connected with the vertical guide rail, and the second driving member is able to drive the vertical lead screw to rotate.

7. The cornea positioning system according to claim 6, wherein the clamping assembly comprises:
a support component, a first clamping component, a second clamping component, a resetting component and a driving component,
wherein a positioning window is disposed at each of central portions of the first clamping component and the second clamping component; the first clamping component is fixed on the support component, the first clamping component and the second clamping component are oppositely disposed, and the first clamping component and the second clamping component are connected via the resetting component therebetween; the drive component comprises a motor, an output shaft of the motor is connected with a swinging arm, and one end of the swinging arm, away from the output shaft of the motor, abuts against the second clamping component; when the output shaft of the motor drives the swinging arm to swing toward the second clamping component, the swinging arm is able to push the second clamping component to rotate toward the first clamping component until the first clamping component touches the second clamping component, and when the output shaft of the motor stops driving the swinging arm to swing, the second clamping component rotates away from the first clamping component under action of the resetting component.

8. The cornea positioning system according to claim 7, wherein
the image recognition system comprises a camera, and a center of the camera is disposed on a perpendicular center line of the positioning screen; and
when the clamping assembly clamps the corneal package body to be between the image recognition system and the background assembly, a center position of the positioning window is located on the perpendicular center line of the positioning screen.

9. The cornea positioning system according to claim 6, wherein one end of the horizontal lead screw is provided with a rotary button, and when the rotary button is rotated, the horizontal lead screw is able to be driven to rotate, making the vertically moving assembly and the clamping assembly move in the horizontal direction.

10. The cornea positioning system according to claim 7, wherein the system further comprises a sample stage, wherein the sample stage is disposed below an initial position of the clamping assembly, a limiting groove is disposed on the sample stage, and the limiting groove is located directly under a gap between the first clamping component and the second clamping component when the first clamping component and the second clamping component are in a clamped state,
wherein the initial position is an initialization state of the clamping component preset on the mechanical arm assembly.

11. A cornea positioning method, applied to a cornea positioning system according to claim 1, comprising:
clamping a corneal package body containing a cornea to-be-positioned via a mechanical arm assembly in an initial position,
moving the corneal package body to be between an image recognition system and a background assembly via the mechanical arm assembly;
providing a background light via the background assembly, and obtaining an image, formed under action of the background light, of the corneal package body via the image recognition system; and
analyzing and calculating the image obtained by the image recognition system via a computing positioning system, to obtain position information of the cornea.

12. The method according to claim 11, wherein the mechanical arm assembly comprises a horizontally moving assembly, a vertically moving assembly and a clamping assembly, wherein the horizontally moving assembly is able to drive the vertically moving assembly and the clamping assembly to reciprocate in a horizontal direction parallel to a surface of a positioning screen; the vertically moving assembly is able to drive the clamping assembly to reciprocate in a vertical direction parallel to the surface of the positioning screen; the horizontally moving assembly comprises a first driving member, a horizontal guide rail, a horizontal lead screw disposed in the horizontal guide rail, and a first nut matching the horizontal lead screw, wherein the first nut is slidably connected with the horizontal guide rail, the first driving member is able to drive the horizontal lead screw to rotate, and the vertically moving assembly is fixedly connected with the first nut; and the vertically moving assembly comprises a second driving member, a vertical guide rail, a vertical lead screw disposed in the vertical guide rail, and a second nut matching the vertical lead screw, wherein the second nut is slidably connected with the vertical guide rail, and the second driving member is able to drive the vertical lead screw to rotate; and
moving the corneal package body to be between the image recognition system and the background assembly via the mechanical arm assembly, comprises:
switching on the second driving member, wherein the second driving member drives the vertical lead screw to rotate to drive the clamping assembly to move vertically from the initial position in a direction parallel to the surface of the positioning screen, and switching off the second driving member when the corneal package body is at a same height as the image recognition system and the background assembly; and
switching on the first driving member, wherein the first driving member drives the horizontal lead screw to rotate to drive the vertically moving assembly and the clamping assembly to move in the horizontal direction parallel to the surface of the positioning screen, and switching off the first driving member when the corneal package body is located between the image recognition system and the background assembly.

13. The method according to claim 12, wherein the clamping assembly comprises a support component, a first clamping component, a second clamping component, a resetting component and a driving component,
wherein a positioning window is disposed at each of central portions of the first clamping component and the second clamping component, the first clamping component is fixed on the support component, the first clamping component and the second clamping component are oppositely disposed, the first clamping component and the second clamping component are connected via the resetting component therebetween, the driving component comprises a motor, an output shaft of the motor is connected with a swinging arm, and one end of the swinging arm, away from the output shaft of the motor, abuts against the second clamping component; and
clamping the corneal package body containing the cornea to-be-positioned via the mechanical arm assembly in the initial position, comprises:
placing and maintaining the corneal package body containing the cornea to-be-positioned between the first clamping component and the second clamping component when the motor is switched off and the second clamping component and the first clamping component are in an open state under action of the resetting component; and
switching on the motor, wherein the output shaft of the motor drives the swinging arm to swing toward the second clamping component, and the swinging arm pushes the second clamping component to rotate toward the first clamping component until the first clamping component touches the second clamping component.

14. The method according to claim 11, wherein the system further comprises a main control system and a mechanical control system,
wherein the mechanical control system is electrically connected with the mechanical arm assembly and configured to control the mechanical arm assembly to move the corneal package body containing the cornea to-be-positioned to be between the image recognition system and the background assembly;
the main control system is electrically connected with the image recognition system and the computing positioning system; is configured to control the image recognition system to obtain the image, formed under the action of the background light, of the corneal package body when the mechanical arm assembly moves the corneal package body to be between the image recognition system and the background assembly; and is configured to control the computing positioning system to analyze and calculate the image obtained by the image recognition system to obtain the position information of the cornea; and
clamping the corneal package body containing the cornea to-be-positioned via the mechanical arm assembly in the initial position comprises:
receiving, through the mechanical control system, a start instruction input by a user;
transmitting, through the mechanical control system, the start instruction input by the user to the main control system;
receiving, through the mechanical control system, a positioning instruction sent by the main control system according to the start instruction;
controlling, through the mechanical control system, the mechanical arm assembly to return to the initial position according to the positioning instruction;
sending, through the mechanical control system, a first signal to the main control system, wherein the first signal is configured to indicate that the mechanical arm assembly has returned to the initial position; and
receiving, through the mechanical control system, a first control signal sent by the main control system, and clamping, by the mechanical control system, the corneal package body containing the cornea in the initial position according to the first control signal.

15. The method according to claim 14, wherein the mechanical arm assembly comprises a horizontally moving assembly, a vertically moving assembly and a clamping assembly, wherein the horizontally moving assembly comprises a first driving member, a horizontal guide rail, a horizontal lead screw disposed in the horizontal guide rail, and a first nut matching the horizontal lead screw, wherein the first nut is slidably connected with the horizontal guide rail, the first driving member is able to drive the horizontal lead screw to rotate, and the vertically moving assembly is fixedly connected with the first nut; and the vertically moving assembly comprises a second driving member, a vertical guide rail, a vertical lead screw disposed in the vertical guide rail, and a second nut matching the vertical lead screw, wherein the second nut is slidably connected with the vertical guide rail, and the second driving member is able to drive the vertical lead screw to rotate; and
controlling, through the mechanical control system, the mechanical arm assembly to return to the initial position according to the positioning instruction comprises:
detecting, through the mechanical control system, a current value of the first nut on the horizontal guide rail and a current value of the second nut on the vertical guide rail;
controlling, through the mechanical control system, the first driving member to be switched on to drive the vertically moving assembly and the clamping assembly to move along the horizontal guide rail to be at a second initial value when the current value of the second nut on the vertical guide rail is a first initial value and the current value of the first nut on the horizontal guide rail is not the second initial value;
controlling, through the mechanical control system, the second nut to drive the clamping assembly to move along the vertical guide rail to be at the first initial value when the current value of the second nut on the vertical guide rail is not the first initial value and the current value of the first nut on the horizontal guide rail is the second initial value;
controlling, though the mechanical control system, the first nut to drive the clamping assembly to move along the horizontal guide rail to be at the second initial value, and controlling, through the mechanical control system, the first nut to drive the clamping assembly to move along the vertical guide rail to be at the first initial value when the current value of the second nut on the vertical guide rail is not the first initial value and the current value of the first nut on the horizontal guide rail is not the second initial value.

16. The method according to claim 15, wherein the clamping assembly comprises:
a support component, a first clamping component, a second clamping component, a resetting component and a driving component,
wherein a positioning window is disposed at each of central portions of the first clamping component and the second clamping component, the first clamping component is fixed on the support component, the first clamping component and the second clamping component are oppositely disposed, the first clamping component and the second clamping component are connected via the resetting component therebetween, the driving component comprises a motor, an output shaft of the motor is connected with a swinging arm, one end of the swinging arm, away from the output shaft of the motor, abuts against the second clamping component, when the output shaft of the motor drives the swinging arm to swing toward the second clamping component, the swinging arm is able to push the second clamping component to rotate toward the first clamping component until the first clamping component touches the second clamping component, and when the output shaft of the motor stops driving the swinging arm to swing, the second clamping component rotates away from the first clamping component under action of the resetting component; and
controlling, through the mechanical control system, the mechanical arm assembly to clamp the corneal package body containing the cornea in the initial position comprises:
controlling, through the mechanical control system, the motor to be switched off, wherein the second clamping component and the first clamping component are in an open state under the action of the resetting component;
placing and maintaining the corneal package body containing the cornea to be between the first clamping component and the second clamping component; and
switching on, through the mechanical control system, the motor at a second time after a preset period, wherein the output shaft of the motor drives the swinging arm to swing toward the second clamping component, the swinging arm pushes the second clamping component to rotate toward the first clamping component until the first clamping component touches the second clamping component.

17. The method according to claim 15, wherein moving the corneal package body to be between the image recognition system and the background assembly via the mechanical arm assembly, comprises:
controlling, through the mechanical control system, the second driving member to be switched on to drive the clamping assembly to slide downward a first distance along the vertical guide rail from the first initial value;
controlling, through the mechanical control system, the second driving member to be switched off after the vertical guide rail slides downward the first distance, making the corneal package body be at a same height as the image recognition system and the background assembly;
controlling, through the mechanical control system, the first driving member to be switched on to drive the vertically moving assembly and the clamping assembly to move a second distance along the horizontal guide rail from the second initial value in a first direction, wherein the first direction is the direction of the horizontal guide rail toward a position between the image recognition system and the background assembly; and
controlling, through the mechanical control system, the first driving member to be switched off after the first nut moves the second distance along the horizontal guide rail in the first direction, making the corneal package body be located between the image recognition system and the background assembly.

18. The method according to claim 14, wherein providing the background light, through the background assembly, and obtaining the image, formed under the action of the background light, of the corneal package body via the image recognition system, and analyzing and calculating the image obtained by the image recognition system via the computing positioning system, to obtain the position information of the cornea, comprises:
controlling, through the main control system, the background assembly to provide the background light, and controlling, by the main control system, the image recognition system to obtain the image, formed under the action of the background light, of the corneal package body; and
controlling, through the main control system, the computing positioning system to analyze and calculate the image obtained by the image recognition system to obtain the position information of the cornea.

19. The method according to claim 18, wherein
after controlling, through the mechanical control system, the mechanical arm assembly to move the corneal package body to be between the image recognition system and the background assembly, before controlling, though the main control system, the computing positioning system to analyze and calculate the image obtained by the image recognition system to obtain the position information of the cornea, the method further comprises:
controlling, through the main control system, the image recognition system and the mechanical control system, making a center position of the positioning window of the clamping assembly be located on the perpendicular center line of the positioning screen.

20. The method according to claim 19, wherein controlling, through the main control system, the image recognition system and the mechanical control system, making the center position of the positioning window of the clamping assembly be located on the perpendicular center line of the positioning screen, comprises:
sending, through the mechanical control system, a second signal to the main control system to indicate that the corneal package body is moved to be between the image recognition system and the background assembly after the mechanical control system controls the mechanical arm assembly to move the corneal package body to be between the image recognition system and the background assembly;
sending, through the main control system, a second control signal to the image recognition system according to the second signal;
detecting, through the image recognition system, a coordinate of the center position of the positioning window according to the second control signal;
matching, through the image recognition system, the coordinate of the center position of the positioning window with a preset center coordinate;
sending, through the image recognition system, deviation information between the coordinate of the center position of the positioning window and the preset center coordinate to the main control system when the coordinate of the center position of the positioning window does not match the preset center coordinate;
sending, through the main control system, the deviation information to the mechanical control system; and
adjusting, through the mechanical control system, the center position of the positioning window according to the deviation information.

21. The method according to claim 14, wherein the method further comprises:
sending, through the computing positioning system, a third signal to the main control system when the computing positioning system analyzes and calculates the image obtained by the image recognition system and determines that there is no cornea related information in the image;
sending, through the main control system, a third control signal to the mechanical control system according to the third signal; and
controlling, through the mechanical control system, the mechanical arm assembly to return to the initial position according to the third control signal.
